(19) United States
(12) Patent Application Publication
(10) Pub. No.: US 2026/0337541 A1
(43) Pub. Date: Oct. 29, 2026

(54) APPARATUS, SYSTEM, AND METHODS FOR FILTERING WASTE IN ROCKER BIOREACTORS

(71) Applicant: GLOBAL LIFE SCIENCES SOLUTIONS USA LLC, MARLBOROUGH, MA (US)

(72) Inventors: Mojtaba PARVIZI, Marlborough, MA (US); Nick MCKENZIE, Marlborough, MA (US); David PRIETO UZCATEGUI, Marlborough, MA (US); William KANE, Marlborough, MA (US); Michael BUCKLEY, Marlborough, MA (US)

(73) Assignee: GLOBAL LIFE SCIENCES SOLUTIONS USA LLC, Marlborough, MA (US)

(21) Appl. No.: 19/110,887

(22) PCT Filed: Sep. 28, 2023
(86) PCT No.: PCT/US2023/033941

§ 371 Date: Mar. 12, 2025

Related U.S. Application Data
(60) Provisional application No. 63/410,821, filed on Sep. 28, 2022.

(51) Int. Cl. C12M 1/00; C12M 3/06

(52) U.S. Cl. CPC C12M 29/10; C12M 23/14; C12M 23/26; C12M 23/40; C12M 27/16; C12M 29/04

(57) ABSTRACT

An apparatus for bioprocessing includes a vessel having a flexible exterior surface defining an interior cavity configured to receive fluid for bioprocessing, the vessel configured for selective attachment to a bioreactor. The vessel includes at least one filter within the interior cavity for retaining cells in the vessel while waste is extracted from the interior cavity and a plurality of waste ports formed on the flexible exterior surface, each of the plurality of waste ports being fluidly connected to the at least one filters to allow waste to be extracted from vessel. The vessel is configured to reduce the likelihood of filter clogging at high cell densities during a bioprocessing procedure in the vessel.

### CROSS-REFERENCE TO RELATED APPLICATIONS

This application is a national stage of International Application No. PCT/US2023/033941 filed on Sep. 28, 2023, which claims the benefit of U.S. Provisional Application Ser. No. 63/410,821, filed on Sep. 28, 2022, all of which are hereby incorporated by reference herein in their entireties.

### BACKGROUND

### Technical Field

Embodiments of the invention relate generally to bioprocessing apparatus, systems, and methods, and, more particularly, to filtering waste from a vessel of a rocker bioreactor during perfusion processing.

### Discussion of Art

Bioreactors are often employed to carry out biochemical and/or biological processes and/or manipulate liquids and other products of such processes. Such bioreactors often include flexible or collapsible vessels, e.g., single-use disposable bags that are supported by an outer rigid structure. These “single-use” bioreactors include stirred tank reactors, in which a sterile disposable bag and agitator are housed within a rigid tank, and rocker bioreactors in which a single-use bag is secured to a rocking platform.

In rocker bioreactors, a disposable bag is secured to a tray which, in turn, is attached to a motorized rocking device. The rocking device causes the tray to pivot back and forth about an axis/pivot point. The motion created by the rocking device induces waves in cell culture media within the bag. The waves provide mixing and gas to liquid transfer of oxygen supplied to the bag, resulting in an ideal environment for cellular growth. To monitor and facilitate cellular growth, rocker bioreactors include, among other features, pumps to add fresh cellular growth/expansion media to the bag and remove used media from the same.

In certain known rocker bioreactors, removal of used media is accomplished via a pump which draws media out of the bag and into a waste receptacle via external tubing connected to a waste port on the bag wall. In perfusion processes, where fresh nutrients are continuously provided while spent media and waste products are simultaneously removed, a filter is utilized to retain cells in the bag during waste removal.

Referring to FIG. 3, such filters, e.g., filter 38, typically rests or floats on top of the cell culture media 32 in the bag 30, and has its own tubing 40, internal to the bag, which connects to the waste port. These filters include a series of small openings or pores on a cell media facing side, which are sized to prevent passage of cells through the filter and out of the bag while allowing spent media and other waste products to be removed. An opposite filter side includes a solid non-porous surface to which the internal tubing 40 is typically formed or attached. In use, a pump applies suction to the filter 38, via an external line connected to the waste port, and waste is withdrawn from the bag 30 while cells are retained in the pores of the filter.

With known bags, however, a possibility of filter clogging at higher cell densities (e.g., those over about 1e7cells/mL) exists. In particular, such filters may clog in and around the area where the internal tubing 40 is formed or attached to the filter 38. This clogging may exist even when other portions of the filter are free from clogging resulting in a filter that is potentially both easily clogged and functionally much smaller than its overall footprint.

As will be appreciated, such clogging may be detrimental to cellular growth as it prevents spent media and other waste products from being removed from the bag. This is generally undesirable as biologics are time consuming to produce and are of high value. Indeed, this may be particularly acute in a cell therapy context where there may be an urgent need for the cellular contents of the bag.

In view of the above, there is a need for a rocker bioreactor vessel that can reduce or prevent the likelihood of clogging of its waste filter particularly at high cell densities during continuous and/or perfusion bioprocesses.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, an apparatus for bioprocessing includes a vessel having a flexible exterior surface defining an interior cavity configured to receive fluid for bioprocessing, the vessel configured for selective attachment to a bioreactor. The apparatus further includes at least one filter within the interior cavity for retaining cells in the vessel while waste is extracted from the interior cavity and a plurality of waste ports formed on the flexible exterior surface, each of the plurality of waste ports being fluidly connected to the at least one filter to allow waste to be extracted from the vessel. The vessel being configured to reduce a likelihood of filter clogging at high cell densities during a bioprocessing procedure in the vessel.

In an embodiment, the bioreactor is a rocker bioreactor and the bioprocessing procedure is a continuous perfusion cell culture process.

In an embodiment, the apparatus of further includes a stop cock manifold fluidly connected to the plurality of waste ports via external waste lines, the stop cock manifold allowing a user to select a non-clogged portion of the at least one filter when another portion of the at least one filter becomes clogged.

In an embodiment, the at least one filter may be a plurality of filters and each of the plurality of waste ports is fluidly connected to a separate filter of the plurality of filters to allow waste to be extracted from the vessel.

In an embodiment, the plurality of filters are separate filter membranes formed on a single piece of material.

In an embodiment, the apparatus further includes a manifold fluidly connected to the plurality of waste ports via external waste lines, the manifold allowing for the simultaneous extraction of waste through each of the plurality of filters.

In an embodiment, the manifold may be a stop cock manifold that allows a user to select a non-clogged filter from the plurality of filters when another filter from the plurality of filters becomes clogged.

In an embodiment, each of the plurality of filters is fluidly connected to a waste port via a filter line located in the interior cavity.

In an embodiment, the plurality of filters may be two filters each of which is fluidly connected to a separate waste port via a filter line.

In an embodiment, the plurality of filters may be four filters each of which is fluidly connected to a separate waste port via a filter line.

In an embodiment, the at least one filter may include pores having a pore size of about 1.2 μm.

In an embodiment, the high cell densities may be greater than about 1e7 cells/mL.

In an embodiment, the plurality of filters are secured within the interior cavity.

According to another aspect of the invention, a system for bioprocessing includes a vessel having a flexible exterior surface defining an interior cavity configured to receive fluid for bioprocessing, the vessel configured for selective attachment to a rocker bioreactor and a plurality of filters within the interior cavity for retaining cells in the vessel during a continuous perfusion cell culture process. The system further includes a plurality of waste ports formed on the flexible exterior surface, the plurality of waste ports being fluidly connected to the plurality of filters via filter lines located within the interior cavity to allow waste to be extracted from the vessel and a manifold fluidly connected to the plurality of waste ports via external waste lines. The system also includes a waste bag fluidly connected to the manifold for receiving waste from the vessel during the continuous perfusion cell culture process. The manifold allowing for the simultaneous extraction of waste through each of the plurality of filters.

In an embodiment, the manifold is a stop cock manifold that allows a user to select a non-clogged filter from the plurality of filters when another filter from the plurality of filters becomes clogged during the continuous perfusion cell culture process.

In an embodiment, the plurality of filters may be a plurality of separate filter membranes formed on a single piece of material.

In an embodiment, the plurality of filters may be two filters that are each fluidly connected to a separate waste port via a filter line.

In an embodiment, plurality of filters may be four filters that are each fluidly connected to a separate waste port via a filter line.

In an embodiment, each of the plurality of filters may include pores having a pore size of about 1.2 μm.

In an embodiment, the high cell densities may be greater than about 1e7 cells/mL.

According to another aspect of the invention, a vessel for bioprocessing includes a vessel having a flexible exterior surface defining an interior cavity configured to receive fluid for bioprocessing, the vessel configured for selective attachment to a bioreactor. The vessel further includes a plurality of filters within the interior cavity for retaining cells in the vessel while waste is extracted from the interior cavity, and a manifold located on the vessel, the manifold having a plurality of filter line ports within the interior cavity and at least one waste port on the flexible exterior surface. The vessel additionally includes a plurality of filter lines fluidly connecting the plurality of filters with the plurality of filter line ports on the manifold to allow waste to be extracted from the vessel through the at least one waste port. The vessel is configured to reduce a likelihood of filter clogging at high cell densities during a bioprocessing procedure in the vessel.

In an embodiment, the manifold may be a stop cock manifold that allows a user to select a non-clogged filter from the plurality of filters if another filter of the plurality of filters becomes clogged at high cell densities during the bioprocessing procedure.

### BRIEF DESCRIPTION OF THE DRAWINGS

The present invention will be better understood from reading the following description of non-limiting embodiments, with reference to the attached drawings, wherein below:

FIG. 1 is a perspective view of a rocker bioreactor system suitable for use with embodiments of the present invention;

FIG. 2 is a perspective view of a known bioreactor vessel used in connection with the rocker bioreactor of FIG. 1, depicting the vessel's filter;

FIG. 3 is a side view of the bioreactor vessel/bag of FIG. 2;

FIG. 4 is a perspective view of a bioreactor vessel and filter according to an embodiment of the present invention;

FIG. 5 is a perspective view of a bioreactor vessel and filter according an alternative embodiment of the present invention;

FIG. 6 is a perspective view of a bioreactor vessel and filters according another embodiment of the present invention;

FIG. 7 is a schematic diagram of a bioreactor vessel, filters, and related equipment according to an embodiment of the invention;

FIG. 8 is a schematic diagram of a bioreactor vessel, filter, and related equipment depicting a four filter and port configuration according to an embodiment of the invention;

FIG. 9 is a schematic diagram of the filter and port configuration of FIG. 8 wherein the filter is mounted on a plate anchored to a bottom vessel surface according to an embodiment of the invention; and

FIG. 10 is a schematic diagram of the filter and port configuration of FIG. 8 wherein the filter tethered to a bottom vessel surface according to another embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and/or “semi-rigid” are used herein interchangeably to describe structures that are “non-collapsible,” that is to say structures that do not fold, collapse, or otherwise deform under normal forces to substantially reduce their elongate dimension.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single-use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid container or vessel used, for example, as a bioreactor or mixer for the contents within. While embodiments of the present invention are described as for use with bioprocessing bags, including but not limited to bioreactor bags and mixer bags, embodiments may also be configured for use with other bags or vessels.

Embodiments may be used to perform a variety of bioprocesses in a rocker bioreactor and are not limited to cellular expansion. Certain embodiments may be broadly used in bioprocessing and biochemical environments, and potentially in non-biological/biochemical contexts. Likewise, while embodiments are described and depicted with respect to specific rocker bioreactor systems and in connection with a continuous perfusion cell culture process, embodiments may potentially be utilized with other types of reactors/mixers, particularly those having vessel in which continuous cell culturing is performed, and/or waste is removed via a filter.

Embodiments are likewise not limited to any specific size/shape of vessel/bag. Though embodiments may be particularly suited and/or described as for use with 20 L-50 L flexible bags, other sizes, e.g., 2 L-10 L, may be utilized.

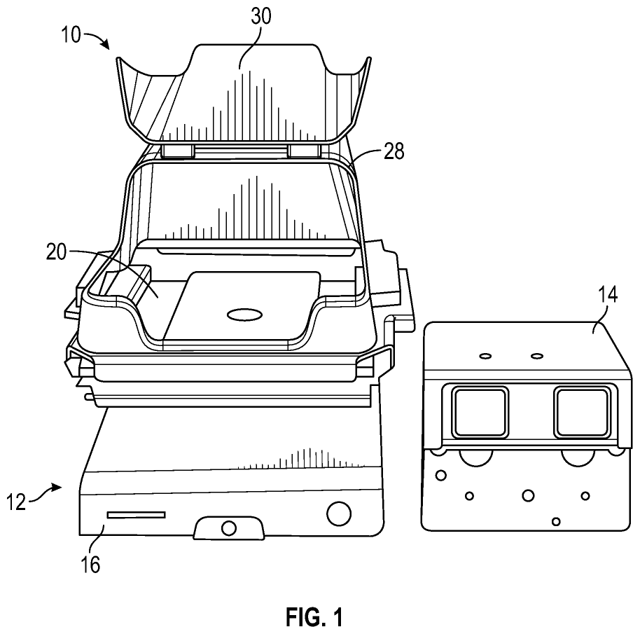

Referring to FIG. 1, an exemplary rocker bioreactor system 10 suitable for use with embodiments of the invention is depicted. As shown, the system 10 includes a bioreactor 12 which is connected to one or more peristaltic pumps 14. The bioreactor 12 is also operatively connected to a controller, e.g., a local or remote computer that provides process/protocol monitoring and the like via a wired or wireless connection (not shown).

The bioreactor 12 includes a removable tray 20, which is configured to selectively receive and support a vessel/bag. In embodiments, the tray 20 includes a removable lid 28 that features a hinged door 30 that may be raised to gain access to the tray 20, and any vessel secured thereto. The bioreactor 12 further includes a base 16 that operatively connected to the tray 20. The base 16 houses a pivoting mechanism, for example, a motor, which enables the tray 20 to pivot back and forth.

In use, a vessel/bag is secured to the tray 20 via one or more attachment mechanisms, e.g., selectively lockable clips located at opposite ends of the tray 20. The tray 20 and bag are then pivoted back and forth creating a rocking motion that induces waves in cell culture media within the bag to facilitate growth.

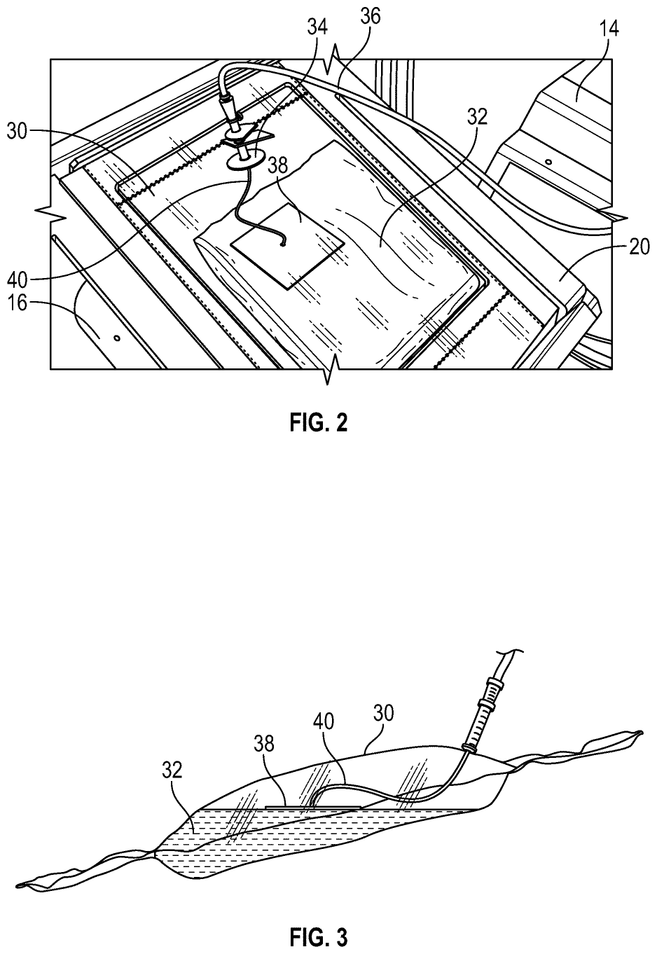

Referring now to FIGS. 2 and 3, a conventional flexible reactor vessel/bag 30 with fluid 32 containing growth medium is depicted. Removal of used media is accomplished via pump 14, e.g., a peristaltic pump, which draws the media out of the bag 30 and into a waste receptacle via an external line/tubing 36 connected to a waste port 34 on the bag wall. To provide perfusion, e.g., retain cells in the bag 30 while spent fluid media 32 is removed, a filter 38 is located within the bag 30. The filter 38 has its own filter line/tubing 40, internal to the bag, which also connects to the waste port 34. With known filters, however, a possibility of clogging at high cell densities, e.g., cell densities greater than about 1e7 cells/mL (1×10⁷).

In particular, filter 38 may clog in and around the area where the internal tubing 40 is formed or attached to the filter 38. This clogging may be present even when other portions of the filter 38 are free from clogging resulting in a filter 38 that is potentially both easily clogged and functionally smaller than its overall footprint. As will be appreciated, such clogging may be detrimental to cellular growth as it prevents spent media and other waste products from being removed from the bag 30.

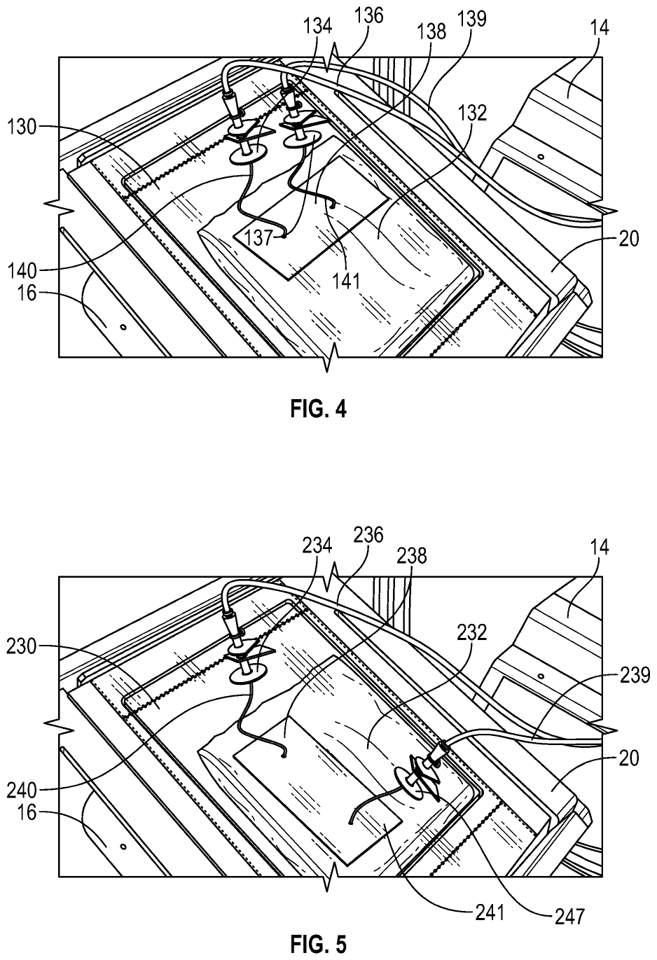

Referring now to FIG. 4, an apparatus for bioprocessing according to an embodiment of the invention is depicted. As shown, the apparatus includes a bag/vessel 130 having a flexible exterior surface that defines an interior cavity that is configured to receive fluid 132, e.g., cell culture media and the like, for bioprocessing. The vessel 130 is configured for selective attachment to a tray 20 of a rocker bioreactor 16.

As shown, the interior cavity of the vessel 130 includes at least one filter 138 and a plurality of waste ports 134, 137. In the depicted embodiment, the filter 138 extends laterally relative to bag 30 and is connected to multiple internal tubes/filter lines 140, 141, and to a plurality of waste ports 134, 137 to which a waste bag (not shown) is fluidly connected via external waste lines 136, 139. As will be appreciated, the filter lines 140, 141 are fluidly connected to the filter 138 to allow waste that has passed through pores on the cell media facing side of the filter (not shown) to exit the vessel 130 via waste ports 134, 137.

The waste ports 134, 137 are formed on the flexible exterior surface of the vessel 130. As will be appreciated, the waste ports 134, 137 may be welded to the vessel 130 or may be molded into or otherwise unitary with the vessel 130. Embodiments are not limited to specific sizes, shapes, or configurations of waste ports 134, 137, however, and conventional ports may be suitable so that commercially available external waste lines 136, 139 and waste bags may be used. The location of the waste ports 134, 137 may vary, though in embodiments they may be located in proximity to (e.g., directly above) where the filter lines 140, 141 are connected to the least one filter 138.

The filter 138 has a cell culture media facing side that includes a plurality of pores (not shown). The filter 138 also has an opposite side, which is solid and pore-less, to which the filter lines 140, 141 are attached or formed. In embodiments, the filter 138 is a laminate or composite of a porous material and a non-porous material with a mesh spacer in between to prevent the porous and non-porous materials from collapsing on one another when suction is applied. The filter lines 140, 141 may be secured to the filters through a variety of methods such as thermal welding or molding.

As will be appreciated, embodiments are not limited to any specific filter materials, constructs, or pore sizes, though, in certain embodiments the filters may be manufactured from a polymer such as polyvinylidene difluoride (PVDF) or polysulfone (PES) and may have a pore size of about 1.2 μm. Likewise, the filter lines 140, 141 are not limited to a specific polymeric material or an inside or outside diameter, length or shape.

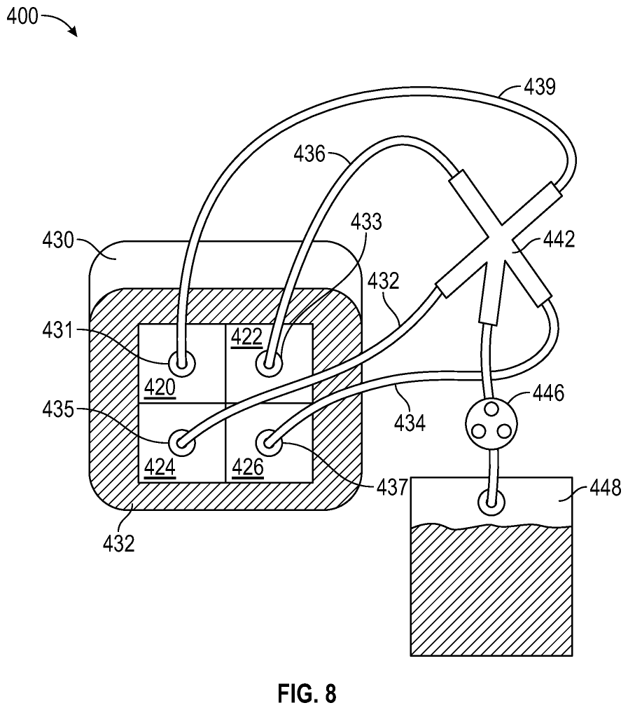

In certain embodiments, the filter 138 may be connected to more than two filter lines. For example, the filter 138 may be connected to filter lines located at each corner of the filter 138 or in an array or other arrangement along the filter 138. Indeed, though embodiments utilize at least two filter lines, they are not limited to only two and number of filter lines may be determined by a variety of factors including the amount of space for corresponding waste ports on the bag's flexible exterior. In specific embodiments, four filter lines (and four waste ports) may be employed (see FIGS. 8-10)

Embodiments are similarly not limited to a specific shape or size of filter, though quadrilateral filters are conventional. As will appreciated, other filter shapes may be utilized without departing from the invention. In embodiments, the filter 138 may be approximately 7″×7″ (17.8 cm×17.8 cm) for 2 L and 10 L vessels/bags, and approximately 15″×15″ (38.1 cm×38.1 cm) for 20 L and 50 L vessels/bags, though other sizes may be utilized.

The vessel/bag itself may be manufactured from a variety of materials and the invention is not limited in this regard. In embodiments, the vessel may be a multilayer laminate of a USP Class VI material. The vessel may be fully or partially transparent to permit viewing of the interior cavity.

Referring again to FIG. 4, the filter lines 140, 141 reduce the likelihood of clogging by providing suction at multiple locations on the filter 138. In this fashion, a larger portion of the filter 138 may be effectively utilized. Moreover, as described in greater detail below, in certain embodiments, one or more of the filter lines 140, 141 may be selectively activated/deactivated via, for example, a stop cock manifold, so a user can select a non-clogged portion of the filter for use when another portion becomes clogged.

Turning now to FIG. 5, an embodiment featuring a longitudinally extending filter 238 may also be utilized. This embodiment is similar to that of FIG. 4 with exception of the orientation of the filter 238 and the location of waste port 247. That is, the apparatus includes a vessel 230 with the filter 238 in the interior cavity. The filter 238 is fluidly connected to waste ports 234, 237 via filter lines 240, 241, respectively. Waste lines 236, 239 run from the waste ports 234, 237 to a waste bag (not shown).

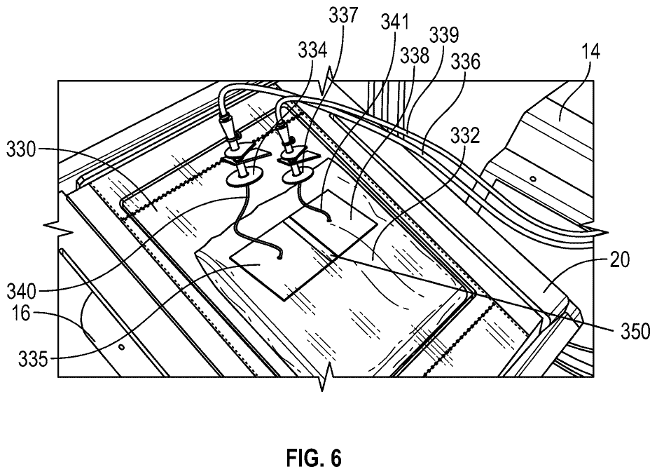

Referring now to FIG. 6 another embodiment of the invention is depicted. In this embodiment, the vessel 330 has a filter that includes a plurality of filters which are separate filter membranes 335, 338 formed on a single piece or sheet of material (e.g., a polymeric laminate). As used herein, the term “filter” includes such filter membranes in addition to physically separate filters. Each filter membrane 335, 338 has pores on its cell culture media facing side (not shown). The filter membranes 335, 338 are separated by a section 350 that effectively isolates or seals each of the filter membranes 335, 338 from the other. This can be accomplished through a variety of methods and, in an embodiment, section 350 is a heat seal between the filter membranes 335, 338.

Each of the filter membranes 335, 338 is connected to its own internal (e.g., first and second) filter lines 340, 341 and waste ports 334, 337. The first and second filter lines 340, 341 utilize separate (e.g., first and second) waste lines 336, 339, which are operatively connected to a pump and waste bag.

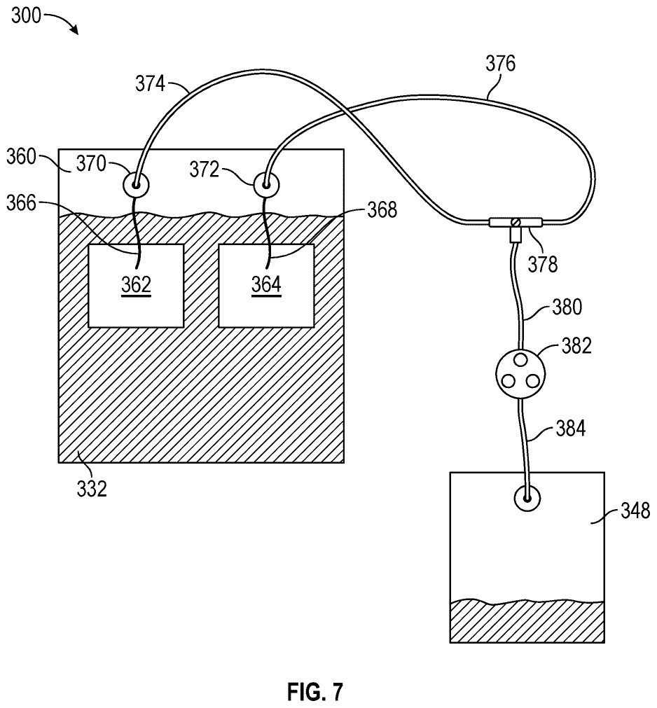

In this embodiment (and the one depicted in FIG. 7) it is envisioned that the filter membranes 335, 338 may be used independently of one another via a stop cock manifold or other switching device. In this respect, a user may select a non-clogged filter from the plurality of filters when another filter from the plurality of filters becomes clogged.

As will be appreciated, in certain embodiments, the plurality of filters may be attached to each other (or to specific filters of the plurality) through other structures or mechanisms. That is, they need not be filter membranes formed from a single sheet of material but may be wholly separate filters that are welded together at certain locations.

Referring now to FIG. 7, a system 300 for bioprocessing includes a vessel 360 with a flexible exterior surface defining an interior cavity. The interior cavity includes a plurality of filters 362, 364. Here, the plurality of filters 362, 364 are two filters that physically separate and not filter membranes on a single sheet of material.

The filters 362, 364 are fluidly connected to a plurality of waste ports 370, 372 via internal filter lines 366, 368. Each filter 362, 364 is connected to a separate waste port via a filter line 366, 368. External waste lines 374 and 376 are connected to the waste ports 370, 372 and are connected to a three-way valve/stop cock manifold 378 which is in turn connected to a peristaltic pump 382 and waste bag 348 via lines/tubing 380, 384. As mentioned, this embodiment allows for swapping a clogged filter with an unused filter mid bioprocessing procedure via the stopcock manifold 342.

Referring now to FIG. 8, in a specific embodiment, a system 400 for bioprocessing includes a vessel 430 with a flexible exterior surface defining an interior cavity that includes a plurality of filters which are four filters, e.g., filter membranes 420, 422, 424, 426, that are formed on a single piece of material, e.g., a polymeric laminate. As with the embodiment of FIG. 6, the filter membranes 420, 422, 424, 426 are isolated from one another by a heat seal or other structure which is represented schematically by the depicted grid. Much like the other embodiments, the filter membranes 420, 422, 424, 426 have a pore containing fluid facing side which contacts the cell media as the filter floats on the same.

In this embodiment, each filter membrane 420, 422, 424, 426 includes a filter line, internal to the vessel, which is not depicted but is configured in accordance with the previously described embodiments. The filter lines, one per filter membrane 420, 422, 424, 426, fluidly connect the membranes to a plurality of waste ports (e.g, four waste ports 431, 433, 435, 437). Each filter membrane 420, 422, 424, 426 is connected to a separate waste port 431, 433, 435, 437 via a filter line. External waste lines 432, 434, 436, 439 are connected to the waste ports 431, 433, 435, 437 and are connected to a four-way manifold 442 which is in turn connected to a peristaltic pump 446 and waste bag 448 via lines/tubing. As will be appreciated, this embodiment allows for simultaneous extraction of waste from all four filter membranes via the manifold 442.

In embodiments, the manifold 442 is a stop cock manifold (or other switching manifold or apparatus) that allows for swapping a clogged filter membrane with an unused or known non-clogged filter membrane mid bioprocessing procedure.

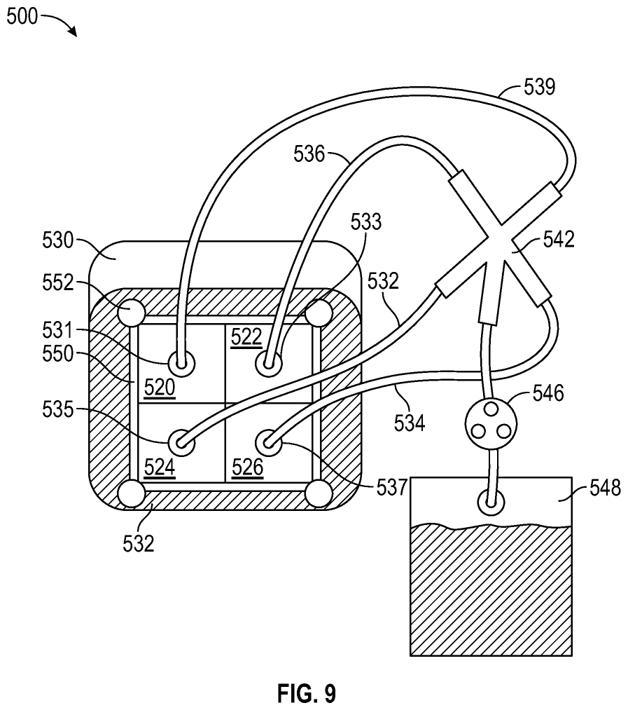

Referring now to FIG. 9, in this embodiment, a system 500 for bioprocessing includes a vessel 530 with a flexible exterior surface defining an interior cavity that includes a plurality of filters which are four filter membranes 520, 522, 524, 526 that are formed on a single piece of material, e.g., a polymeric laminate. As with the embodiment of FIG. 8, the filter membranes 520, 522, 524, 526 are isolated from one another by a heat seal or other structure represented schematically by the depicted grid. Again, the filter membranes 520, 522, 524, 526 have a pore containing fluid facing side which contacts the cell media as the filter floats on the same.

Each filter membrane 520, 522, 524, 526 includes a filter line which fluidly connects the membranes to a plurality of waste ports (e.g, four waste ports 531, 533, 535, 537), external waste lines 532, 534, 536, 539, a four-way manifold 542, which in embodiments may be a stop cock manifold, a pump 546, and to a waste bag 548 via lines/tubing. With this embodiment, however, the filter membranes 520, 522, 524, 526 are secured to a rigid plate 552 which is anchored to a bottom surface of the vessel.

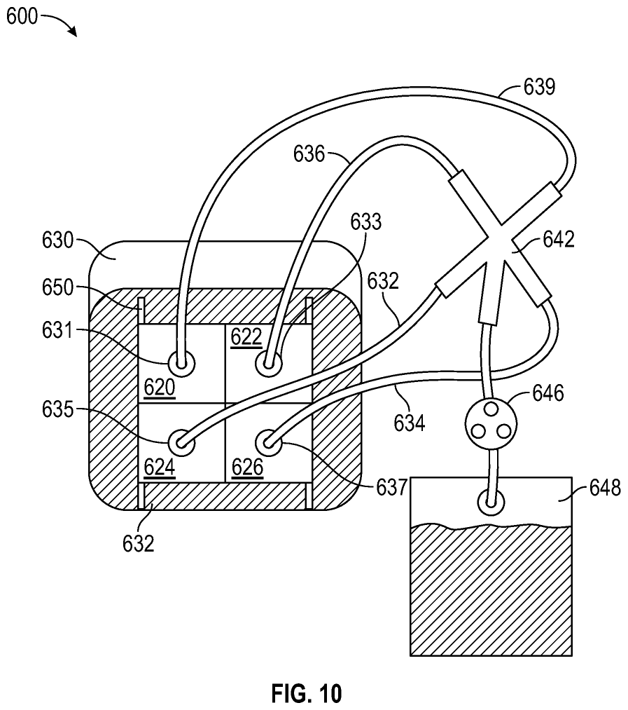

Similarly, as shown in FIG. 10, a system 600 bioprocessing includes a vessel 630 which includes a four filter membranes 620, 622, 624, 626 that are formed on a single piece of material and include a filter line which fluidly connects the membranes to a plurality of waste ports (e.g, four waste ports 631, 633, 635, 637), external waste lines 632, 634, 636, 639, a four-way manifold 642 (e.g., stop cock manifold), a pump 646, and to a waste bag 648 via lines/tubing. In this embodiment, however, the filter membranes 620, 622, 624, 626, while not mounted to a rigid plate as with the embodiment of FIG. 9, are secured to a bottom surface of the vessel via a plurality of tethers. The tethers may be formed from the same single piece of material as the filter membranes and/or may be welded to the bottom surface of the vessel. Of course, other attachment mechanisms may be employed without departing from the scope of the invention.

Although embodiments are described for use in connection with manifolds and stop cock manifolds generally a variety of manifolds or switching mechanisms/valve banks may be utilized. These include pneumatic valves and the like. In embodiments, such manifolds/switching mechanisms may be operatively connected to a controller.

In a specific embodiment, the vessel may have a manifold located on the vessel itself. In such embodiments, the manifold may be located on (e.g., formed in or otherwise attached to) the flexible exterior surface of the vessel such that a plurality of filter line ports of the manifold would be located within the interior cavity and at least one waste port would be located on the flexible exterior surface. In such embodiments, a plurality of filters within the interior cavity would be fluidly connected to the plurality of filter line ports via a plurality of filter lines to allow waste to be extracted from the vessel through the at least one waste port. In embodiments, the manifold may be a stop cock manifold. As will be appreciated, in such embodiments, there would be no need for an external manifold and the external waste line would be connected to a pump and waste bag.

In embodiments that feature a plurality of filters/filter membranes, the cumulative surface area of the filters/filter membranes may total the cumulative surface area of a single 7″×7″ or 15″×15″ filter.

In certain embodiments, the selection of a filter and flow of spent media/waste into the waste receptacle may be automated or otherwise electronically controlled. For example, a flow sensor (not shown) located on, e.g., an external waste line 374, 376 may detect a clogged filter (or filter membrane) and may send an alert to a user and/or automatically shut off the afflicted filter and select another yet to be used (or known not clogged) filter from those within the interior cavity of the vessel. In embodiments, the selection of a filter via a stop cock or manifold may be remotely operated by a user via a smart device or other controller or may be scheduled/preprogrammed. That is, the filters may be activated in an alternating sequence to prevent clogging.

In this regard, embodiments of the invention also contemplate a method of filtering waste in a rocker bioreactor. In an aspect, the method includes the step of initiating a bioprocessing procedure, e.g., cell culture, in a vessel having an interior cavity with a plurality of filters. The method further includes determining if one or more of the plurality of filters is clogged. If a filter is clogged, the method includes selecting and activating an unclogged filter so that waste may be extracted from the vessel. In certain embodiments, the method also includes periodically selecting, activating, and deactivating different filters from the plurality to prevent future filter clogging. The method may also include an initial step of activating all filters present in the vessel to maximize waste removal.

As mentioned, in embodiments, method steps may be automated, scheduled, or preprogrammed via, for example, a plurality of sensors and a controller. In other embodiments, a user can manually perform the inventive method steps.

As used herein, an element or step recited in the singular and proceeded with the word “a” or “an” should be understood as not excluding plural of said elements or steps, unless such exclusion is explicitly stated. Furthermore, references to “one embodiment” or “an embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

[symbol]

What is claimed is:

1. An apparatus for bioprocessing comprising:
a vessel having a flexible exterior surface defining an interior cavity configured to receive fluid for bioprocessing, the vessel configured for selective attachment to a bioreactor;
at least one filter within the interior cavity for retaining cells in the vessel while waste is extracted from the interior cavity; and
a plurality of waste ports formed on the flexible exterior surface, each of the plurality of waste ports being fluidly connected to the at least one filter to allow waste to be extracted from the vessel;
wherein the vessel is configured to reduce a likelihood of filter clogging at high cell densities during a bioprocessing procedure in the vessel.

2. The apparatus of claim 1 wherein the bioreactor is a rocker bioreactor and the bioprocessing procedure is a continuous perfusion cell culture process.

3. The apparatus of claim 1 further comprising:
a stop cock manifold fluidly connected to the plurality of waste ports via external waste lines, the stop cock manifold allowing a user to select a non-clogged portion of the at least one filter when another portion of the at least one filter becomes clogged.

4. The apparatus of claim 1 wherein the at least one filter is a plurality of filters and each of the plurality of waste ports is fluidly connected to a separate filter of the plurality of filters to allow waste to be extracted from the vessel.

5. The apparatus of claim 4 wherein the plurality of filters are separate filter membranes formed on a single piece of material.

6. The apparatus of claim 4 further comprising:
a manifold fluidly connected to the plurality of waste ports via external waste lines, the manifold allowing for simultaneous extraction of waste through each of the plurality of filters.

7. The apparatus of claim 6 wherein the manifold is a stop cock manifold that allows a user to select a non-clogged filter from the plurality of filters when another filter from the plurality of filters becomes clogged.

8. The apparatus of claim 4 wherein each of the plurality of filters is fluidly connected to a waste port via a filter line located in the interior cavity.

9. The apparatus of claim 4 wherein the plurality of filters are two filters each of which is fluidly connected to a separate waste port via a filter line.

10. The apparatus of claim 4 wherein the plurality of filters are four filters each of which is fluidly connected to a separate waste port via a filter line.

11. The apparatus of claim 1 wherein the at least one filter includes pores having a pore size of about 1.2 μm.

12. The apparatus of claim 1 wherein the high cell densities are greater than about 1e7 cells/mL.

13. The apparatus of claim 1 wherein plurality of filters are secured within the interior cavity.

14. A system for bioprocessing comprising:
a vessel having a flexible exterior surface defining an interior cavity configured to receive fluid for bioprocessing, the vessel configured for selective attachment to a rocker bioreactor;
a plurality of filters within the interior cavity for retaining cells in the vessel during a continuous perfusion cell culture process;
a plurality of waste ports formed on the flexible exterior surface, the plurality of waste ports being fluidly connected to the plurality of filters via filter lines located within the interior cavity to allow waste to be extracted from the vessel;
a manifold fluidly connected to the plurality of waste ports via external waste lines; and
a waste bag fluidly connected to the manifold for receiving waste from the vessel during the continuous perfusion cell culture process;
wherein the manifold allowing for simultaneous extraction of waste through each of the plurality of filters.

15. The system of claim 14 wherein the manifold is a stop cock manifold that allows a user to select a non-clogged filter from the plurality of filters if another filter of the plurality of filters becomes clogged at high cell densities during the continuous perfusion cell culture process.

16. The system of claim 14 wherein the plurality of filters is a plurality of separate filter membranes formed on a single piece of material.

17. The system of claim 14 wherein the plurality of filters are two filters that are each fluidly connected to a separate waste port via a filter line.

18. The system of claim 14 wherein the plurality of filters are four filters that are each fluidly connected to a separate waste port via a filter line.

19. The system of claim 14 wherein each of the plurality of filters includes pores having a pore size of about 1.2 μm.

20. The system of claim 15 wherein the high cell densities are greater than about 1e7 cells/mL.

21. A vessel for bioprocessing comprising:
a vessel having a flexible exterior surface defining an interior cavity configured to receive fluid for bioprocessing, the vessel configured for selective attachment to a bioreactor;
a plurality of filters within the interior cavity for retaining cells in the vessel while waste is extracted from the interior cavity;
a manifold located on the vessel, the manifold having a plurality of filter line ports within the interior cavity and at least one waste port on the flexible exterior surface; and
a plurality of filter lines fluidly connecting the plurality of filters with the plurality of filter line ports on the manifold to allow waste to be extracted from the vessel through the at least one waste port;
wherein the vessel is configured to reduce a likelihood of filter clogging at high cell densities during a bioprocessing procedure in the vessel. 22. The vessel of claim 21 wherein the manifold is a stop cock manifold that allows a user to select a non-clogged filter from the plurality of filters if another filter of the plurality of filters
becomes clogged at high cell densities during the bioprocessing procedure.
